(19) United States
(12) Patent Application Publication
(10) Pub. No.: US 2017/0266218 A1
(43) Pub. Date: Sep. 21, 2017

(54) NAD+ COENZYME FORMULATION AND METHODS OF MAKING AND USING THE SAME

(71) Applicant: Steve McNerlin, Birmingham (GB)

(72) Inventor: Steve McNerlin, Birmingham (GB)

(21) Appl. No.: 15/076,462

(22) Filed: Mar. 21, 2016

(51) Int. Cl. A61K 31/7084; A61K 8/60; A61Q 7/00; A61K 9/00

(52) U.S. Cl. CPC A61K 31/7084; A61K 9/0043; A61K 9/0019; A61K 8/606; A61Q 7/00

(57) ABSTRACT

Embodiments of the present invention disclose formulations comprising NAD for reducing sugar craving and methods of making and using the same. Embodiments of the invention also discloses dosage forms comprising the formulation of invention and a delivery device such as nasal spray, nasal inhaler, or injection device.

### FIELD OF THE INVENTION

The present invention provides a novel formulation for reducing or ameliorating sugar craving and a related disorder.

### BACKGROUND OF THE INVENTION

The stimulation of sweet taste causes a feeling of pleasantness as a consequence of stimulating dopamine in the brain. However, it has been shown that the repeated ingestion of sugar can lead to a craving or addiction syndrome. Animal studies demonstrated that test animals develop features similar to that observed with drug addiction, and will show signs of anxiety or withdrawal following elimination of sugar from the diet or the administration of naloxone (Nakagawa T, et al., American journal of physiology. 2006 March; 290 (3):F625-31; Perez-Pozo S E, et al., Int J Obes (Lond). 2009 Dec. 22). The mechanism appears to relate to a reduction in dopamine receptors in the nucleus accumbens from chronic dopamine stimulation, leading to a loss of control mechanisms in the frontal and prefrontal cortex (Roncal C A, et al., American journal of nephrology. 2009 Aug. 21; 30 (5):399-404).

Additionally, excessive sugar intake has been linked to many medical conditions such as obesity, diabetic conditions, and other metabolic and non-metabolic conditions.

To date, formulations for sugar craving reduction is limited. Excessive sugar consumption continues to be a major factor that leads to the prevalence of metabolic conditions. As such, there is a need for additional formulations and methods for reducing sugar craving.

The embodiments described below address the above identified issues and need.

### SUMMARY OF THE INVENTION

In one aspect of the present invention it is provided a method for reducing sugar craving or treating or ameliorating a related condition in a mammal, said method comprising administering to said mammal a composition comprising an effective amount of nicotinamide adenine dinucleotide (NAD) in a form where net NAD+ is >0 to reduce the sugar craving or treating or ameliorating the condition in the mammal.

In some embodiments of the invention method, optionally in combination with any or all of the various embodiments disclosed herein, administering comprises administration of the composition by nasal administration.

In some embodiments of the invention method, optionally in combination with any or all of the various embodiments disclosed herein, administering comprises administration of the composition by nasal spray or nasal inhaling.

In some embodiments of the invention method, optionally in combination with any or all of the various embodiments disclosed herein, administering comprises administration of the composition by scalp injection.

In some embodiments of the invention method, optionally in combination with any or all of the various embodiments disclosed herein, the mammal is of a metabolic condition.

In some embodiments of the invention method, optionally in combination with any or all of the various embodiments disclosed herein, the related condition is one of obesity, a metabolic syndrome, hair loss, aging, sleep disorder, a renal disease, cancer, Alzheimer's disease or coronary condition.

In another aspect of the present invention, it is provided a formulation comprising an effective amount of nicotinamide adenine dinucleotide (NAD) in a form where net NAD+ is >0 to reduce the sugar craving or treating or ameliorating the condition in the mammal.

In some embodiments of the invention formulation, optionally in combination with any or all of the various embodiments disclosed herein, the formulation is in a dosage form for nasal administration.

In some embodiments of the invention formulation, optionally in combination with any or all of the various embodiments disclosed herein, the formulation is a dry power formulation.

In some embodiments of the invention formulation, optionally in combination with any or all of the various embodiments disclosed herein, the formulation further comprises a liquid carrier.

In some embodiments of the invention formulation, optionally in combination with any or all of the various embodiments disclosed herein, the formulation is in a dosage form selected from a paste, ointment, or a suspension of particles in a liquid carrier.

In a further aspect of the present invention, it is provided a kit, comprising:

a formulation effective for reducing sugar craving treating or ameliorating a related condition in a mammal,

a dosing unit that provides one or more doses of the formulation, each dose providing an effective amount of nicotinamide adenine dinucleotide (NAD) in a form where net NAD+ is >0 to reduce the sugar craving or treating or ameliorating the condition in the mammal,

an optional liquid housing unit for housing an optional liquid carrier, and

a dispensing unit for dispensing the formulation in the dosing unit for application to a subject in need thereof,

wherein the dosage unit, the optional liquid housing unit, and the dispensing unit are separate, partially joined, or entirely joined forming a single structure.

In some embodiments of the invention kit, optionally in combination with any or all of the various embodiments disclosed herein, the application is nasal administration.

In some embodiments of the invention kit, optionally in combination with any or all of the various embodiments disclosed herein, the liquid carrier is water.

In some embodiments of the invention kit, optionally in combination with any or all of the various embodiments disclosed herein, the formulation is a powder formulation.

In some embodiments of the invention kit, optionally in combination with any or all of the various embodiments disclosed herein, the formulation is a paste, ointment, or a suspension of particles in a liquid carrier.

In some embodiments of the invention kit, optionally in combination with any or all of the various embodiments disclosed herein, the related condition is one of obesity, a metabolic syndrome, hair loss, aging, sleep disorder, a renal disease, cancer, Alzheimer's disease or coronary condition.

In a further aspect of the present invention, it is provided a method, comprising:

providing a formulation effective for reducing sugar craving treating or ameliorating a related condition in a mammal,

providing a dosing unit that provides one or more doses of the formulation, each dose providing an effective amount of nicotinamide adenine dinucleotide (NAD) in a form where net NAD+ is >0 to reduce the sugar craving or treating or ameliorating the condition in the mammal,

providing an optional liquid housing unit for housing an optional liquid carrier,

providing a dispensing unit for dispensing the formulation in the dosing unit for application to a subject in need thereof, and

forming the kit;

wherein the dosage unit, the optional liquid housing unit, and the dispensing unit are separate, partially joined, or entirely joined forming a single structure.

In some embodiments of the invention method, optionally in combination with any or all of the various embodiments disclosed herein, the application is nasal administration.

In some embodiments of the invention method, optionally in combination with any or all of the various embodiments disclosed herein, the liquid carrier is water.

In some embodiments of the invention method, optionally in combination with any or all of the various embodiments disclosed herein, the formulation is a powder formulation.

In some embodiments of the invention method, optionally in combination with any or all of the various embodiments disclosed herein, the formulation is a paste, ointment, or a suspension of particles in a liquid carrier.

In some embodiments of the invention method, optionally in combination with any or all of the various embodiments disclosed herein, the related condition is one of obesity, a metabolic syndrome, hair loss, aging, eye sight, a renal disease, cancer, or coronary condition.

### BRIEF DESCRIPTION OF THE DRAWINGS

FIG. 1A shows the baseline hair condition of patient 1 before using an embodiment formulation of the invention formulation.

FIG. 1B shows the hair condition of patient 1 after using an embodiment formulation of the invention formulation at the last review.

FIG. 2A shows the baseline hair condition of patient 2 before using an embodiment formulation of the invention formulation.

FIG. 2B shows the hair condition of patient 2 after using an embodiment formulation of the invention formulation at the last review.

FIG. 3A shows the baseline hair condition of patient 3 before using an embodiment formulation of the invention formulation.

FIG. 3B shows the hair condition of patient 3 after using an embodiment formulation of the invention formulation at the last review.

FIG. 4A shows the baseline hair condition of patient 4 before using an embodiment formulation of the invention formulation.

FIG. 4B shows the hair condition of patient 4 after using an embodiment formulation of the invention formulation at the last review.

FIG. 5A shows the baseline hair condition of patient 5 before using an embodiment formulation of the invention formulation.

FIG. 5B shows the hair condition of patient 5 after using an embodiment formulation of the invention formulation at the last review.

FIG. 6A shows the baseline hair condition of patient 6 before using an embodiment formulation of the invention formulation.

FIG. 6B shows the hair condition of patient 6 after using an embodiment formulation of the invention formulation at the last review.

FIG. 7 shows an embodiment of the invention kit.

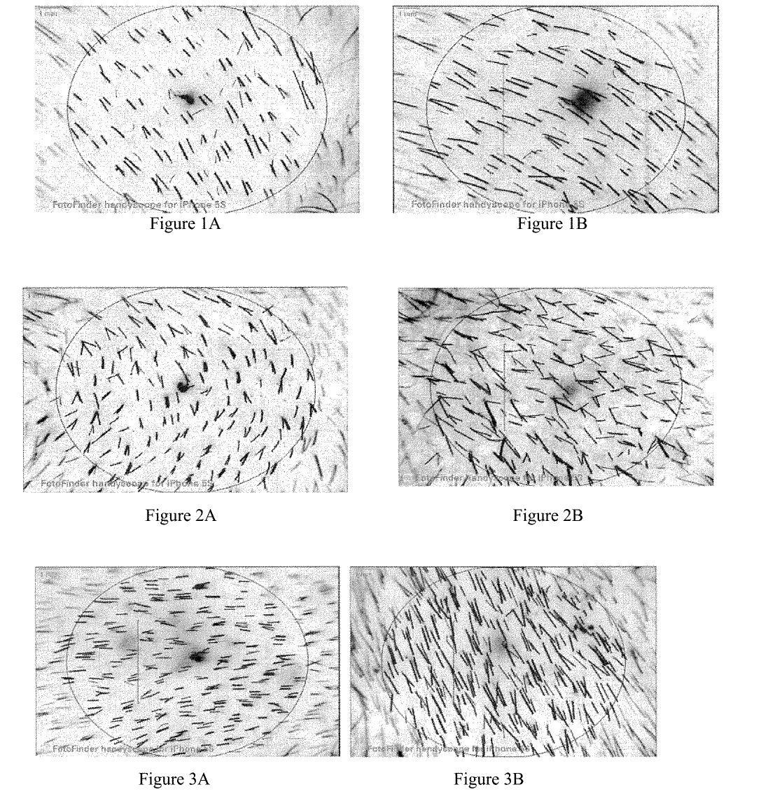

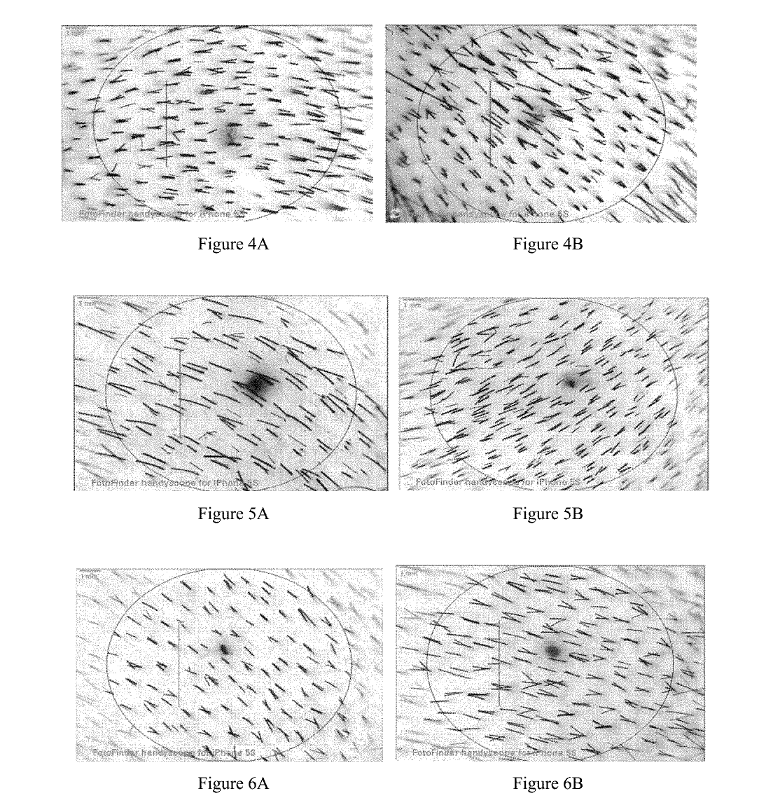

### DETAILED DESCRIPTION OF THE INVENTION

### Definitions

As used herein, the terms “administering” or “administration” of an agent, drug, or peptide to a subject includes any route of introducing or delivering to a subject a compound to perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another.

As used herein, the terms “diabetic” or “diabetes” refers to Type 1 diabetes, wherein the pancreas produces little or no insulin; Type 2 diabetes, wherein the body becomes resistant to the effects of insulin or produces little or no insulin; or disease state occurring as sequelae of other primary diseases that include the symptoms of either or both of elevated blood sugar (hyperglycemia) and the excretion of sugar in the urine (glycosuria).

As used herein, the terms “disease,” “disorder,” or “complication” refers to any deviation from a normal state in a subject. In preferred embodiments, the methods and compositions of the present invention are useful in the diagnosis and treatment of diseases where the expression of a KHK protein differs between subjects with disease and subjects not having disease. The present invention finds use with any number of diseases including, but not limited to, renal diseases.

As used herein, by the term “effective amount,” “amount effective,” “therapeutically effective amount,” or the like, it is meant an amount effective at dosages and for periods of time necessary to achieve the desired result.

As used herein, a “formulation,” “pharmaceutical formulation” all include a composition comprising at least one of NAD+ or a precursor thereof. Optionally, the “composition,” “pharmaceutical composition” or “therapeutic agent” further comprises pharmaceutically acceptable diluents or carriers.

As used herein, the term “preventing” means causing the clinical symptoms of the disease state not to develop, e.g., inhibiting the onset of disease, in a subject that may be exposed to or predisposed to the disease state, but does not yet experience or display symptoms of the disease state.

As used herein, the term “subject” refers to any animal (e.g., a mammal), including, but not limited to, humans, non-human primates, rodents, and the like, which is to be the recipient of a particular treatment.

As used herein, the terms “treating” or “treatment” or “alleviation” refers to both therapeutic treatment and prophylactic or preventative measures, wherein the object is to prevent or slow down (lessen) the targeted pathologic condition or disorder.

As used herein, the terms “composition” and “formulation” are sometimes used interchangeably.

### Nicotinamide Adenine Dinucleotide

Nicotinamide adenine dinucleotide (NAD) is a coenzyme found in all living cells. The compound is a dinucleotide, because it consists of two nucleotides joined through their phosphate groups. One nucleotide contains an adenine base and the other nicotinamide. Nicotinamide adenine dinucleotide exists in two forms, an oxidized and reduced form abbreviated as NAD+ and NADH respectively, as shown below:

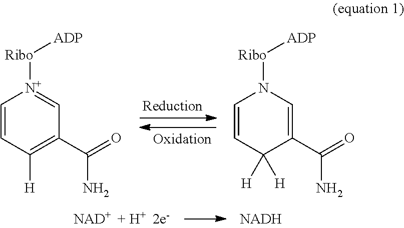

In metabolism, nicotinamide adenine dinucleotide is involved in redox reactions, carrying electrons from one reaction to another. The coenzyme is, therefore, found in two forms in cells: NAD+ is an oxidizing agent—it accepts electrons from other molecules and becomes reduced. This reaction forms NADH, which can then be used as a reducing agent to donate electrons. These electron transfer reactions are the main function of NAD. However, it is also used in other cellular processes, the most notable one being a substrate of enzymes that add or remove chemical groups from proteins, in posttranslational modifications. Because of the importance of these functions, the enzymes involved in NAD metabolism are targets for drug discovery.

In organisms, NAD can be synthesized from simple building-blocks (de novo) from the amino acids tryptophan or aspartic acid. In an alternative route, more complex components of the coenzymes are taken up from food as vitamin niacin. Similar compounds are released by reactions that break down the structure of NAD. These preformed components then pass through a salvage pathway that recycles them back into the active form. Some NAD is also converted into nicotinamide adenine dinucleotide phosphate (NADP); the chemistry of this related coenzyme is similar to that of NAD, but it has different roles in metabolism.

Although NAD+ is written with a superscript plus sign because of the formal charge on a particular nitrogen atom, at physiological pH for the most part it is actually a singly charged anion (charge of minus 1), while NADH is a doubly charged anion.

### The Redox Reactions of Nicotinamide Adenine Dinucleotide.

In metabolism, the compound NAD accepts or donates electrons in redox reactions. Such reactions (summarized in formula below) involve the removal of two hydrogen atoms from the reactant (R), in the form of a hydride ion (H−), and a proton (H+). The proton is released into solution, while the reductant RH2 is oxidized and NAD+ reduced to NADH by transfer of the hydride to the nicotinamide ring.

RH2+NAD+NADH+H++R;

From the hydride electron pair, one electron is transferred to the positively charged nitrogen of the nicotinamide ring of NAD+, and the second hydrogen atom transferred to the C4 carbon atom opposite this nitrogen. The midpoint potential of the NAD+/NADH redox pair is −0.32 volts, which makes NADH a strong reducing agent. The reaction is easily reversible, when NADH reduces another molecule and is re-oxidized to NAD+. This means the coenzyme can continuously cycle between the NAD+ and NADH forms without being consumed.

### Functions

Nicotinamide adenine dinucleotide has several essential roles in metabolism. It acts as a coenzyme in redox reactions, as a donor of ADP-ribose moieties in ADP-ribosylation reactions, as a precursor of the second messenger molecule cyclic ADP-ribose, as well as acting as a substrate for bacterial DNA ligases and a group of enzymes called sirtuins that use NAD+ to remove acetyl groups from proteins. In addition to these metabolic functions, NAD+ emerges as an adenine nucleotide that can be released from cells spontaneously and by regulated mechanisms (Smyth L M, et al., J Biol Chem. 279 (47): 48893-903 (2004); Billington R A, et al., Mol Med. 12 (11-12): 324-7 (2006)) and can therefore have important extracellular roles (Id.)

### Net NAD+ Value

Applicant of the instant application found that NAD+ is therapeutically effective for the uses disclosed herein whereas NADH is entirely inactive. As such, Applicant believes NAD+ alone is responsible for the activities disclosed herein. On the other hand, as substrate of enzymes, NADH often competes against NAD+ for binding to the enzymes, and thus, in this sense, inactive NADH would at least partially neutralize the activities of NAD+ as a substrate where it competes with NADH for binding to the enzymes. Applicant therefore defines NADH as having a negative net NAD+ value. In this context, as used herein, the term “NAD+ precursor” refers to a compound that generates NAD+ but does not neutralize the effect NAD+, and as such, a “NAD+ precursor” does not include NADH nor Nacin.

### Regulation of SIRT1 by NAD+

While the functions of NAD+ are the focus of many studies, it is believed the mechanism of action of NAD+ with respect to the invention disclosed herein is its ability to modulate the level of SIRT1. A decrease of SIRT1 level, in turn, reportedly is linked to many disorders, briefly described below:

### a) Fatty Liver

You et al. reported that alcohol inhibits or decreases SIRT1 expression in alcohol fatty liver disease (AFLD) patients (You, M., et al., HeptatoBilary Surg. Nutr. 2015; 4(2):88-100). A new study led by Boston University School of Medicine (BUSM) demonstrates that the abnormal metabolism linked to obesity could be regulated in part by the interaction of two metabolic regulators, called the NAD-dependent deacetylase SIRT1 and fibroblast growth factor 21 (FGF21). Using experimental models, the researchers found that a lack of SIRT1 protein in the liver led to lower levels of a liver secreted protein FGF21, which resulted in an increased likelihood of developing fatty liver disease and obesity (Zhang, Mengwei, et al., Gastroenterology, 2014, 146(2):539-549)).

### b) Aging-Associated Disorders

A lower level of SIRT1 is found to be associated with aging-associated disorders such as Alzheimer's and weight gain (see, e.g., Ng, F., et al., SIRT1 in the brain—connections with aging-associated disorders and lifespan, in Frontiers in Cellular Neuroscience, March 2015, vol. 9, Article 64 (Review). As context, the starvation hormone, fibroblast growth factor-21 (FGF21), was found to effectively extend the lifespan in mice (Zhang et al., eLife 2012; 1:e00065). The metabolic effect of FGF21, e.g., anti-obesity and glucose lowering, was reviewed by Kharitonenkov and Adams in Molecular Metabolism 3 (2014) 221-229.

### c) Aging and Hair Loss

Hair greying and hair loss and coronary diseases are generally regarded as signs of aging, Geyfman and Andersen, (Albany, N.Y.), 2010 March; 2(3):122-128). Interestingly, male pattern baldness was found to be associated with coronary heart disease (Yamada, T., et al, BMJ Open 2013; 3:e002537);

### d) Heart Failure and Coronary Diseases

Akkafa et al. recently reported that reduced SIRT1 expression correlates with enhanced oxidative stress in compensated and decompensated heart failure (Akkafa, F., et al., Redox Biology 6:169-173 (2015).

### e) Age-Associated Weight Gain

A literature survey by Sasaki (Sasaki Tsutomu, Age-associated weight gain, leptin, and SIRT1: a possible role for hypothalamic SIRT1 in the prevention of weight gain and aging through modulation of leptin sensitivity, in Frontiers in Endocrinology, Vol 6, Article 109 (2015) (Review) provide an account of the role of SIRT1 in the prevention of weight gain in aging).

### f) Retinal Aging and Degeneration

Mimura et al. reported that SIRT1 has retinal neuroprotective effect (Mimura, T., et al., JSM Ophthalmol 2(1):1016 (2014)).

On the other hand, NAD has been reported to reverse aging in that it enables communication inside cells between the nucleus and mitochondria (https://hms.harvard.edu/news/genetics/new-reversible-cause-aging-12-19-13). A study reported in 2012 there exists a link between oxidative stress and PARP activity, aging, and a decline in NAD+ levels in human tissue (Massudi, H., et al., PLoS One, July 2012, Vol. 7 (7):e42357). NAD's ability to restore cell communication to reverse the aging process is also confirmed in another report (https://www.theguardian.com/science/2013/dec/20/anti-ageing-human-trials).

As used herein, while the term “NAD” is sometimes used instead of NAD+, the reference to NAD in the instant application shall mean the net amount of NAD+ in the NAD is higher than NADH in that the molar ratio of NAD+/NADH in the NAD >1.

### Pharmaceutical Compositions/Formulation

Therefore, in another aspect of the present invention, it is provided a formulation for reducing sugar craving, comprising an effective amount of nicotinamide adenine dinucleotide (NAD+) or a precursor thereof and a carrier for nasal administration or scalp injection.

In some embodiments of the invention formulation, optionally in combination with any or all the various embodiments of invention formulation, the nasal administration is nasal spray or nasal inhaling.

In some embodiments of the invention formulation, optionally in combination with any or all the various embodiments of invention formulation, the carrier is a liquid carrier.

In some embodiments of the invention formulation, optionally in combination with any or all the various embodiments of invention formulation, the carrier is a powder carrier.

In some embodiments of the invention formulation, optionally in combination with any or all the various embodiments of invention formulation, the formulation is a liquid formulation, a dry powder formulation, a paste, ointment, or a suspension of particles in a liquid carrier.

The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, these compositions can contain suitable pharmaceutically acceptable carriers comprising excipients and auxiliaries which facilitate processing of the active compounds into preparations which can be used pharmaceutically. Compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Further details on techniques for formulation and administration can be found in the latest edition of REMINGTON'S PHARMACEUTICAL SCIENCES (Maack Publishing Co., Easton, Pa., which is incorporated herein by reference). After pharmaceutical compositions have been prepared, they can be placed in an appropriate container and labeled for treatment of an indicated condition. Such labeling would include amount, frequency, and method of administration.

It is noted that the compositions and methods disclosed herein may be administered to any subject as defined herein. In one embodiment, the subject is human. In another embodiment, the subject is a pet, e.g., cat, dog, or the like, and in a particular embodiment, is an overweight pet or animal. It is further noted that a corresponding composition, e.g., a pharmaceutical composition, may be provided for use in any method described herein.

All forms of NAD coenzyme are white amorphous powders that are hygroscopic and highly water-soluble. The solids are stable if stored dry and in the dark. Solutions of NAD+ are colorless and stable for about a week at 4° C. and neutral pH, but decompose rapidly in acids or alkalis. Upon decomposition, they form products that are enzyme inhibitors. Various formulations therefore can be made making use of such properties of NAD.

Those skilled in the art will appreciate that numerous delivery mechanisms are available for delivering a therapeutic agent to an area of need. By way of example, the agent may be delivered using a liposome as the delivery vehicle. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human.

### Method of Use

In one aspect of the present invention, it is provided a method for diminishing, inhibiting or eliminating sugar craving in a mammal, wherein said method comprises administering to said mammal an effective amount of a composition comprising nicotinamide adenine dinucleotide (NAD+) or a precursor thereof to reduce the sugar craving in the mammal.

In some embodiments of the invention method, optionally in combination with any or all the various embodiments of invention method, administering comprises administration of the composition by nasal administration.

In some embodiments of the invention method, optionally in combination with any or all the various embodiments of invention method, administering comprises administration of the composition by nasal spray or nasal inhaling.

In some embodiments of the invention method, optionally in combination with any or all the various embodiments of invention method, administering comprises administration of the composition by scalp injection.

In some embodiments of the invention method, optionally in combination with any or all the various embodiments of invention method, the mammal is of a metabolic condition.

In some embodiments of the invention method, optionally in combination with any or all the various embodiments of invention method, the metabolic condition is of obesity, metabolic syndrome, or renal disease.

Other medical conditions that can be treated or ameliorated using the composition of invention include any disorders associated with aging, for example, hair loss, male pattern alopecia, female pattern alopecia, skin, fatty liver, overweight, diabetic conditions, macular degeneration, retinal disease or degeneration, ADD (attention deficient disorder), ADHD (attention deficient hyperactivity disorder), Alzheimer's disease, cancer, UV damaged skin, coronary disease, and heart failure.

### Dosage Form

In another aspect of the present invention, it is provided a dosage form, comprising a dosage module comprising a formulation for reducing sugar craving and a delivery device, the formulation comprising an effective amount of nicotinamide adenine dinucleotide (NAD+) or a precursor thereof and a carrier for nasal delivery or scalp injection, and the delivery device is a nasal spray device or injection device.

Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration; drug combination(s), reaction sensitivities, and tolerance/response to therapy. Long-acting compositions can be administered every 3 to 4 days, every week, or once every two weeks depending on the half-life and clearance rate of the particular formulation.

In some embodiments, dosage amount can be determined in reference to concentration and state of NAD in living cells. It is reported that in rat liver, the total amount of NAD+ and NADH is approximately 1μ mole per gram of wet weight, about 10 times the concentration of NADP+ and NADPH in the same cells (Reiss P D, et al., Anal. Biochem. 140 (1): 162-7 (1984)). The actual concentration of NAD+ in cell cytosol is harder to measure, with recent estimates in animal cells, ranging around 0.3 mM (Yamada K, et al., Anal. Biochem. 352 (2): 282-5 (2006); Yang H, et al., Cell 130 (6): 1095-107 (2007)) and approximately 1.0 to 2.0 mM in yeast (Belenky P, et al., Cell 129 (3): 473-84) (2007). However, more than 80% of NADH fluorescence in mitochondria is from bound form, so the concentration in solution is much lower (Blinova K, et al., Biochemistry 44 (7): 2585-94) (2005).

Delivery devices can be any device capable of delivering NAD to a subject via a given mode of delivery. Examples of such devices include, but are not limited to, nasal spray device or nasal inhaler for nasal admission, and injection devices. In some embodiments, the device can be a mesolithal injection device.

### Method of Fabricating Formulation

In a further aspect of the present invention, it is provided a method of forming a formulation for reducing sugar craving, comprising providing an effective amount of nicotinamide adenine dinucleotide (NAD+) or a precursor thereof and a carrier for nasal delivery or scalp injection, and forming the formulation.

In some embodiments of the invention method, optionally in combination with any or all the various embodiments of invention method, the carrier is a carrier for nasal administration.

In some embodiments of the invention method, optionally in combination with any or all the various embodiments of invention method, the carrier for nasal administration is a liquid carrier.

In some embodiments of the invention method, optionally in combination with any or all the various embodiments of invention method, the carrier is a powder carrier.

### A Kit

In a further aspect of the present invention, it is provided a kit, comprising:

a formulation effective for reducing sugar craving treating or ameliorating a related condition in a mammal,

a dosing unit that provides one or more doses of the formulation, each dose providing an effective amount of nicotinamide adenine dinucleotide (NAD) in a form where net NAD+ is >0 to reduce the sugar craving or treating or ameliorating the condition in the mammal,

an optional liquid housing unit for housing an optional liquid carrier, and

a dispensing unit for dispensing the formulation in the dosing unit for application to a subject in need thereof,

wherein the dosage unit, the optional liquid housing unit, and the dispensing unit are separate, partially joined, or entirely joined forming a single structure.

In some embodiments of the invention kit, optionally in combination with any or all of the various embodiments disclosed herein, the application is nasal administration.

In some embodiments of the invention kit, optionally in combination with any or all of the various embodiments disclosed herein, the liquid carrier is water.

In some embodiments of the invention kit, optionally in combination with any or all of the various embodiments disclosed herein, the formulation is a powder formulation.

In some embodiments of the invention kit, optionally in combination with any or all of the various embodiments disclosed herein, the formulation is a paste, ointment, or a suspension of particles in a liquid carrier.

In some embodiments of the invention kit, optionally in combination with any or all of the various embodiments disclosed herein, the related condition is one of obesity, a metabolic syndrome, hair loss, aging, sleep disorder, a renal disease, cancer, Alzheimer's disease or coronary condition.

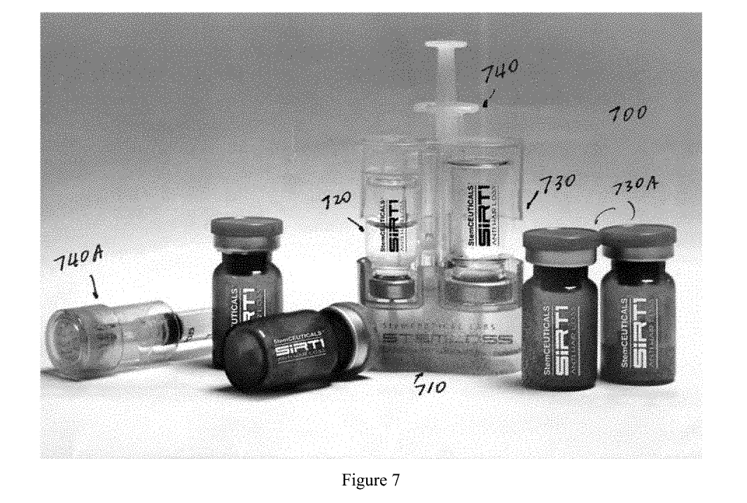

FIG. 7 illustrates a kit of the one embodiment of the present invention. FIG. 7, kit 700 includes a base 710, which provides a base for a liquid carrier 720 in a container and a NAD+ formulation 730 or 730A in a container where the liquid carrier 720 and the NAD+ formulation can be made connected such that a pressure difference in the two can cause the liquid carrier to be transferred to the NAD+ formulation container so as to make a solution of NAD+ formulation 730 in liquid carrier 720. The solution can be transferred to a mesolithal injection device 740 or 740A for mesolithal application. Alternatively, an applicator (e.g., a nasal sprayer) can be attached to the container of the solution to allow one to conveniently use the solution.

### Method of Fabricating the Kit

In a further aspect of the present invention, it is provided a method, comprising:

providing a formulation effective for reducing sugar craving treating or ameliorating a related condition in a mammal,

providing a dosing unit that provides one or more doses of the formulation, each dose

providing an effective amount of nicotinamide adenine dinucleotide (NAD) in a form where net NAD+ is >0 to reduce the sugar craving or treating or ameliorating the condition in the mammal,

providing an optional liquid housing unit for housing an optional liquid carrier,

providing a dispensing unit for dispensing the formulation in the dosing unit for application to a subject in need thereof, and

forming the kit;

wherein the dosage unit, the optional liquid housing unit, and the dispensing unit are separate, partially joined, or entirely joined forming a single structure.

In some embodiments of the invention method, optionally in combination with any or all of the various embodiments disclosed herein, the application is nasal administration.

In some embodiments of the invention method, optionally in combination with any or all of the various embodiments disclosed herein, the liquid carrier is water.

In some embodiments of the invention method, optionally in combination with any or all of the various embodiments disclosed herein, the formulation is a powder formulation.

In some embodiments of the invention method, optionally in combination with any or all of the various embodiments disclosed herein, the formulation is a paste, ointment, or a suspension of particles in a liquid carrier.

In some embodiments of the invention method, optionally in combination with any or all of the various embodiments disclosed herein, the related condition is one of obesity, a metabolic syndrome, hair loss, aging, eye sight, a renal disease, cancer, or coronary condition.

The following examples illustrate rather than limit the embodiments of the present invention.

### EXAMPLES

### Example 1

### Database Trial Studies on the Evaluation of the SIRT1 Enzyme in Follicular Stimulation

### Introduction:

There is an emerging interest in the role of the SIRT 1 enzyme in the context of the complex hair growth cycle. This interest has been elevated as investigators have reported that the SIRT1 enzyme controls the role of androgen receptor (AR) function, and also recently published data of macrophages which had a positive effect on hair growth in animal studies, and SIRT1 inhibits the inflammatory pathways in macrophages, both functions have been found to play prominent roles in Alopecia.

The repression of DHT requires the NAD-dependent catalytic function of SIRT1, which provides a direct functional link between the AR and SIRT1. Macrophages have their inflammatory pathway controlled by SIRT1. Also the Prostaglandin PDG2 is also inhibited by unregulated SIRT1.

### Method:

All six patients underwent weekly micro injections to the subcutaneous fatty layer of the affected layer of the scalp using 4 ml of a SIRT1 Activator (NAD+) over a 24 week period using a commercially available Biophymed CE certified U225 mesotherapy device. The SIRT 1 activator was specially prepared from our written protocol.

### Results:

Patients were followed for safety and tolerability as well as for monitoring differences in hair growth. In order to track progress over time, we employed global photography, and a 60 second patient observational assessment tool based on a standardized 60 second hair count protocol, haircount and trichoscan analysis (Fotofinder); Trichoscan analysis allows us to quantitatively track the following parameters: hair count, hair density, anagen/telogen rates, along with cumulative hair thickness Patients were asked to return to the clinic at 6 weeks, 12 weeks after the intervention. Our early experience demonstrates an encouraging trend in 12 week trichoscan parameters, showing an average of 21% increase in hair count.

### Conclusion:

Preliminary results are statistically significant and positive. Various formulations are being developed, including an IV infusion product as well as the development of a capsule product that will stand the passage through stomach into the gastrointestinal tract where it's release will circumvent the need for micro and IV treatments.

### Example 2

### Inhibition of Sugar Craving

Overweight patients suffering from sugar carving were administered a NAD+ formulation (data not shown) via i.v. 7 hours/day. After 10 treatments, the sugar craving was gone in each of the patients receiving the treatment. Detailed data is not shown.

### Example 3

### Weight Reduction

Overweight Guinea pigs were administered a formulation of NAD+ (data not shown) for 3 weeks. Weight loss was observed. An average weight loss in an average guinea pig was observed, corresponding to about 15 lbs weight loss in an average human being at the time point of three weeks and about 20 lbs at the time point 6 weeks. The positive response rate is 100%. Detailed data is not shown.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

The teachings of the references, including patents and patent related documents, cited herein are incorporated herein in their entirety to the extent not inconsistent with the teachings herein.

I claim:

1. A method for reducing sugar craving or treating or ameliorating a related condition in a mammal, said method comprising administering to said mammal a composition comprising an effective amount of nicotinamide adenine dinucleotide (NAD) in a form where net NAD+ is >0 to reduce the sugar craving or treating or ameliorating the condition in the mammal.

2. The method of claim 1, wherein administering comprises administration of the composition by nasal administration.

3. The method of claim 1, wherein administering comprises administration of the composition by nasal spray or nasal inhaling.

4. The method of claim 1, wherein administering comprises administration of the composition by scalp injection.

5. The method of claim 1, wherein the mammal is of a metabolic condition.

6. The method of claim 5, wherein the related condition is one of obesity, a metabolic syndrome, hair loss, aging, sleep disorder, a renal disease, cancer, Alzheimer's disease or coronary condition.

7. A formulation comprising an effective amount of nicotinamide adenine dinucleotide (NAD) in a form where net NAD+ is >0 to reduce the sugar craving or treating or ameliorating the condition in the mammal.

8. The formulation of claim 7 in a dosage form for nasal administration.

9. The formulation of claim 7, which is a dry power formulation.

10. The formulation of claim 7, further comprising a liquid carrier.

11. The formulation of claim 7 in a dosage form selected from a paste, ointment, or a suspension of particles in a liquid carrier.

12. A kit, comprising:
a formulation effective for reducing sugar craving treating or ameliorating a related condition in a mammal,
a dosing unit that provides one or more doses of the formulation, each dose providing an effective amount of nicotinamide adenine dinucleotide (NAD) in a form where net NAD+ is >0 to reduce the sugar craving or treating or ameliorating the condition in the mammal,
an optional liquid housing unit for housing an optional liquid carrier, and
a dispensing unit for dispensing the formulation in the dosing unit for application to a subject in need thereof,
wherein the dosage unit, the optional liquid housing unit, and the dispensing unit are separate, partially joined, or entirely joined forming a single structure.

13. The kit of claim 12, wherein the application is nasal administration.

14. The kit of claim 12, wherein the liquid carrier is water.

15. The kit of claim 12, wherein the formulation is a powder formulation.

16. The kit of claim 12, wherein the formulation is a paste, ointment, or a suspension of particles in a liquid carrier.

17. The kit of claim 12, wherein the related condition is one of obesity, a metabolic syndrome, hair loss, aging, sleep disorder, a renal disease, cancer, Alzheimer's disease or coronary condition.

18. A method, comprising:
providing a formulation effective for reducing sugar craving treating or ameliorating a related condition in a mammal,
providing a dosing unit that provides one or more doses of the formulation, each dose providing an effective amount of nicotinamide adenine dinucleotide (NAD) in a form where net NAD+ is >0 to reduce the sugar craving or treating or ameliorating the condition in the mammal,
providing an optional liquid housing unit for housing an optional liquid carrier,
providing a dispensing unit for dispensing the formulation in the dosing unit for application to a subject in need thereof, and
forming the kit;
wherein the dosage unit, the optional liquid housing unit, and the dispensing unit are separate, partially joined, or entirely joined forming a single structure.

19. The method of claim 18, wherein the application is nasal administration.

20. The method of claim 18, wherein the liquid carrier is water.

21. The method of claim 18, wherein the formulation is a powder formulation.

22. The method of claim 18, wherein the formulation is a paste, ointment, or a suspension of particles in a liquid carrier.

23. The method of claim 18, wherein the related condition is one of obesity, a metabolic syndrome, hair loss, aging, sleep disorder, a renal disease, cancer, Alzheimer's disease or coronary condition.
